(19) United States
(12) Patent Application Publication
(10) Pub. No.: US 2010/0324441 A1
(43) Pub. Date: Dec. 23, 2010

(54) Brain-Related Chronic Pain Disorder Treatment Method and Apparatus

(76) Inventors: Jeffrey B. Hargrove, Bancroft, MI (US); Robert M. Ford, Troy, MI (US); Buckley D. Beranek, Greenwood, IN (US)

Correspondence Address: REISING ETHINGTON P.C., P O BOX 4390, TROY, MI, 48099-4390, US

(21) Appl. No.: 12/865,286

(22) PCT Filed: Jan. 30, 2009
(86) PCT No.: PCT/US09/32639

§ 371 Date: Jul. 29, 2010

Related U.S. Application Data
(63) Continuation of application No. 12/187,375, filed on Aug. 6, 2008.
Application No. 12/187,375 is a continuation-in-part of application No. 11/490,255, filed on Jul. 21, 2006, now Pat. No. 7,715,910.
Application No. 11/490,255 is a continuation of application No. 10/357,503, filed on Feb. 4, 2003, now abandoned.
(60) Provisional application No. 61/024,641, filed on Jan. 30, 2008.
(60) Provisional application No. 60/963,486, filed on Aug. 6, 2007.
(60) Provisional application No. 61/014,917, filed on Dec. 19, 2007.
(60) Provisional application No. 61/024,641, filed on Jan. 30, 2008.
(60) Provisional application No. 61/032,241, filed on Feb. 28, 2008.
(60) Provisional application No. 60/353,234, filed on Feb. 4, 2002.

(30) Foreign Application Priority Data
Aug. 6, 2008 (US) PCT/US2008/072389
Aug. 6, 2008 (US) PCT/US2008/072395
Dec. 18, 2008 (US) PCT/US2008/087451

(51) Int. Cl. A61B 5/04

(52) U.S. Cl. 600/544; 607/46

(57) ABSTRACT

A method for treating brain-related chronic pain disorders in human subjects includes assessing the brain function of a subject suffering from chronic pain, diagnosing a chronic pain-related abnormal brain condition, and mitigating the abnormal brain activity by applying an electrical stimulation signal to tissues corresponding to at least one area of abnormal brain activity.

### CROSS-REFERENCES TO RELATED APPLICATIONS

This application claims priority in PCT/US2008/072395 filed 6 Aug. 2008, U.S. Ser. No. 12/187,375 filed 6 Aug. 2008, PCT/US2008/087451 18 Dec. 2008 and PCT/US2008/072389, 6 Aug. 2008, all of which are incorporated herein by reference in their entirety.

### TECHNICAL FIELD

This invention relates generally to a method and apparatus for treating brain-related chronic pain disorders in human subjects.

### BACKGROUND OF THE INVENTION

Few methods are known in the prior art for treating brain-related chronic pain disorders such as fibromyalgia. U.S. Pat. No. 7,146,205 issued 5 Dec. 2006 to Holman, discloses a fibromyalgia treatment method including the use of inhibitors of sympathetic nervous system activities. U.S. Pat. No. 5,990,162, issued 23 Nov. 1999 to Scharf, discloses a fibromyalgia treatment method that involves the use of butyrate derivatives. U.S. Pat. No. 5,378,686, issued 3 Jan. 1995 to Bennett, discloses a fibromyalgia treatment involving the use of supplemental growth hormone. In each of these documents the inventors teach methods that likely address central nervous system and neurotransmitter outcomes that result from the fundamental role of brain function in the pathology of fibromyalgia. However, the methods disclosed in these patents are unable to treat the fundamental brain function abnormality related to fibromyalgia that causes chronic pain.

In addition, United States Patent Application Publication No. 20070191905 published 16 Aug. 2007; Nos. 20070179563 and 20070179564 published 2 Aug. 2007, No. 20070156182 published 5 Jul. 2007, No. 20070106339 published 10 May 2007, and No. 20070106342 published 10 May 2007, disclose the use of electrical stimulation to treat hypotension, vision disorders, dysphagia, and pain, respectively. However, none of the methods disclosed in these publications are able to treat a brain function abnormality that is at least partially responsible for causing chronic pain.

What is needed is a method and apparatus for treating brain-related chronic pain disorders in human subjects that can treat a fundamental brain function abnormality associated with chronic pain.

### SUMMARY OF THE INVENTION

A method for treating a brain-related chronic pain disorder in a human subject is provided. According to this method one can treat a brain-related chronic pain disorder in a human subject by assessing the brain function of a subject suffering from chronic pain, diagnosing a chronic pain-related abnormal brain condition, and mitigating the abnormal brain activity by applying an electrical stimulation signal to tissues corresponding to the at least one area of abnormal brain activity.

### BRIEF DESCRIPTION OF THE DRAWINGS

These and other features and advantages of the invention will become apparent to those skilled in the art in connection with the following detailed description, drawings, photographs, and appendices, in which:

FIG. 1A is a flow chart depicting a method performed according to the invention;

FIG. 1B is a continuation of the flow chart of FIG. 1A;

FIG. 1C is a continuation of the flow chart of FIG. 1B;

FIG. 1D is a continuation of the flow chart of FIG. 1C;

FIG. 1E is a continuation of the flow chart of FIG. 1D;

FIG. 2 is a schematic diagram showing an apparatus for treating a brain-related chronic pain disorder according to the invention;

FIG. 3 is a schematic diagram showing a signal generating circuit of the apparatus of FIG. 2;

FIG. 4 is a schematic diagram showing an embodiment of an apparatus for treating a brain-related chronic pain disorder according to the invention and showing a therapy cap of the apparatus and a subject's head cut-away to reveal electrical stimulation signal paths relative to target areas of the subject's brain tissue;

FIG. 5 is a schematic diagram showing an embodiment of an apparatus for treating a brain-related chronic pain disorder according to the invention and showing a therapy cap of the apparatus cut-away to reveal an RFID chip carried by the cap; and

FIG. 6 is a schematic diagram showing an embodiment of an apparatus for treating a brain-related chronic pain disorder according to the invention and showing a therapy cap of the apparatus cut-away to reveal a programmable memory circuit carried by the cap.

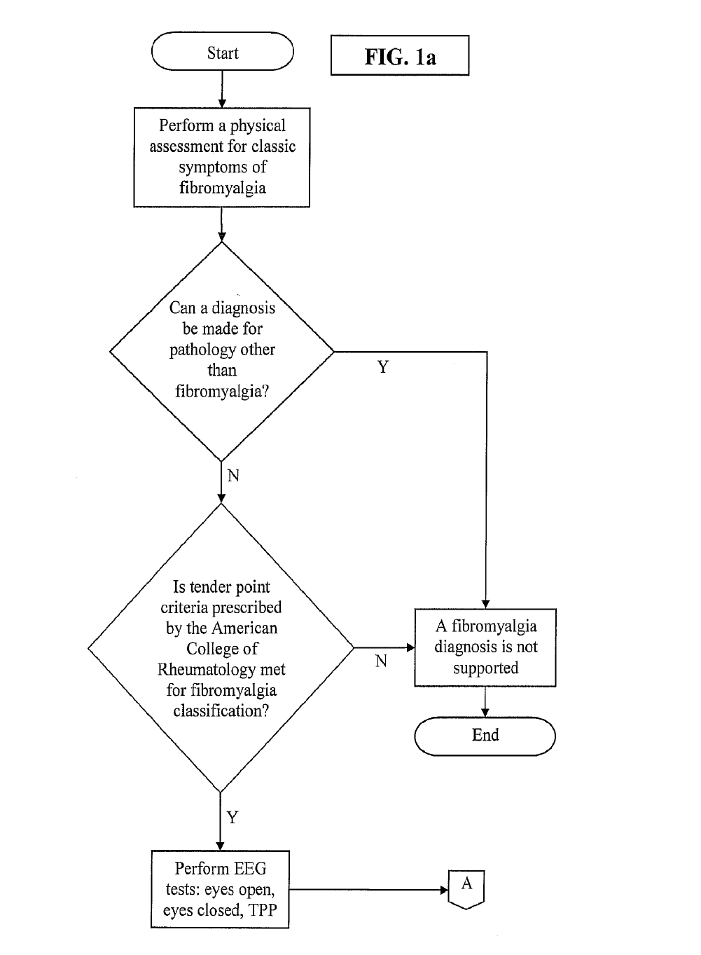

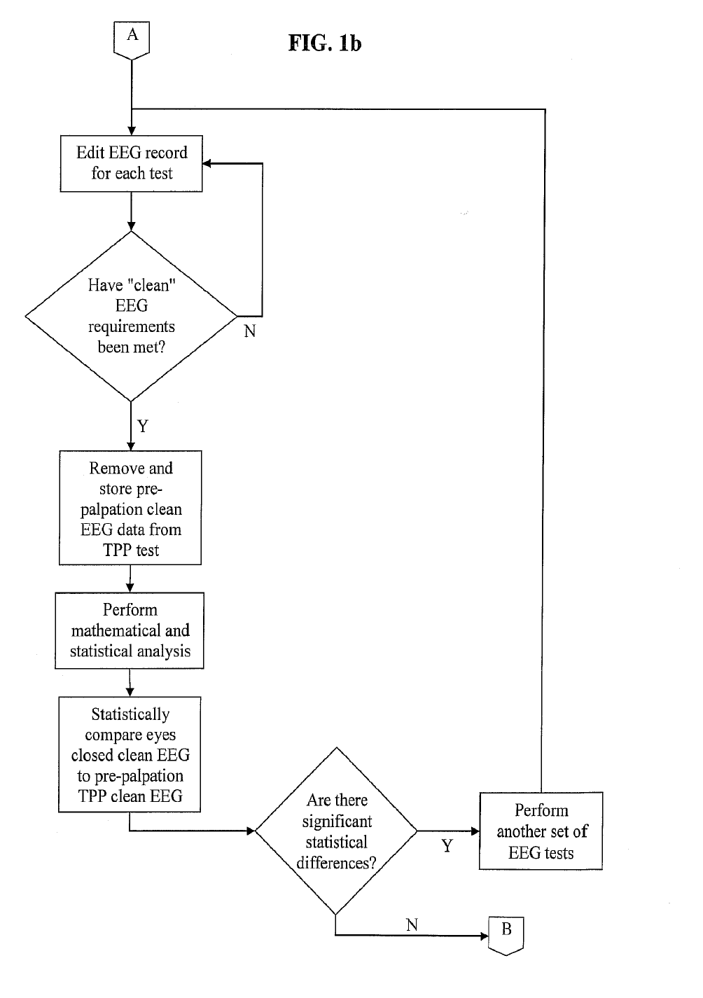

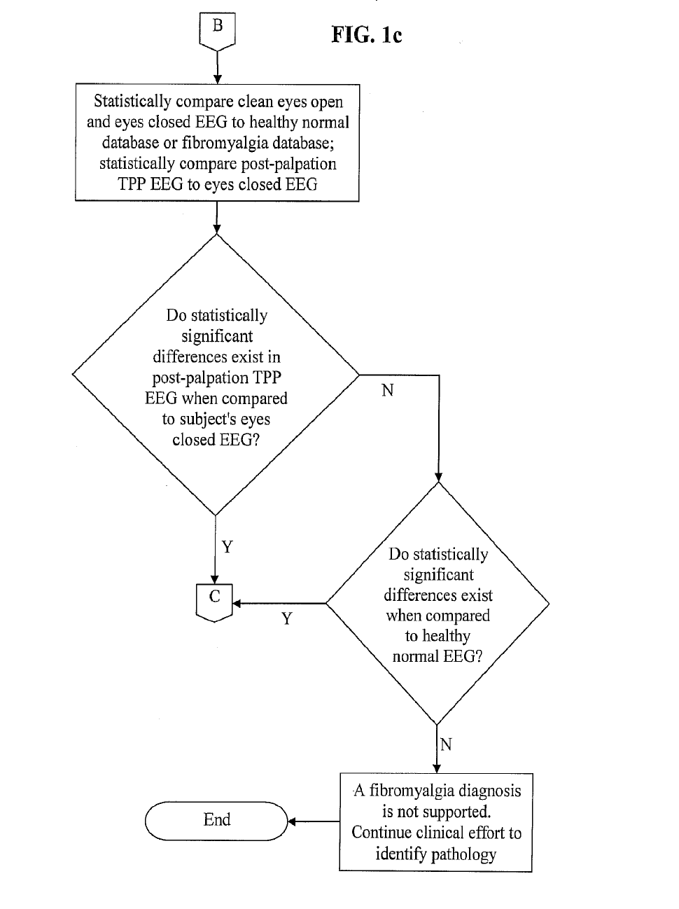

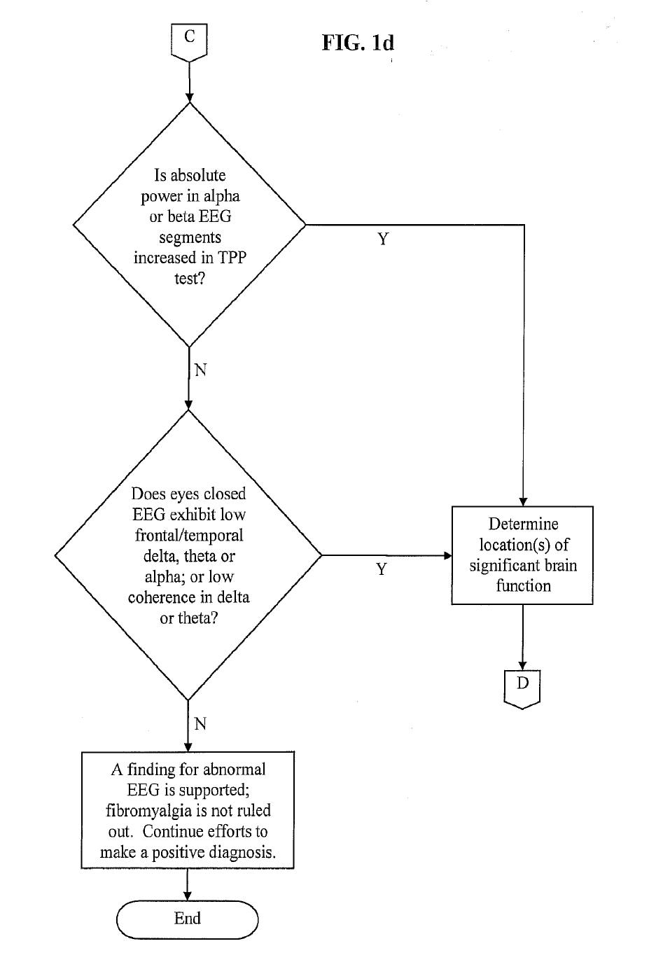

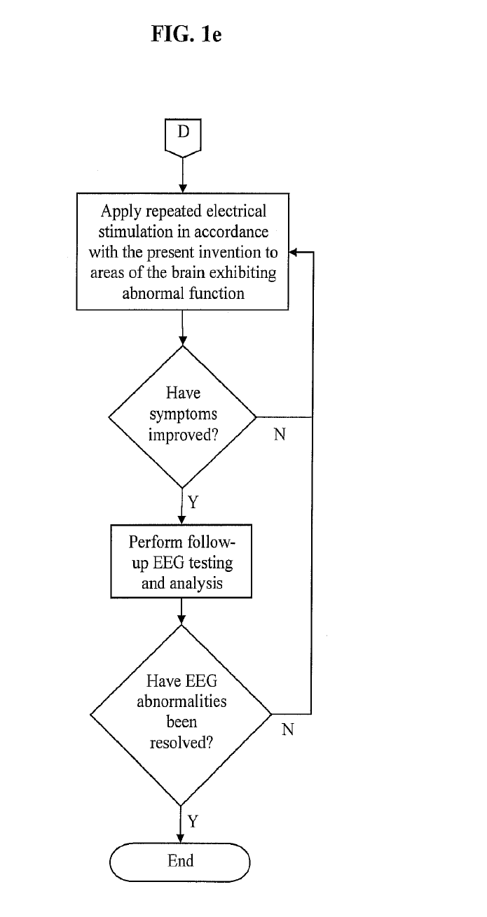

### DETAILED DESCRIPTION OF INVENTION EMBODIMENT(S)

A method is provided for treating a brain-related chronic pain disorder. The method includes assessing the brain function of a subject suffering from chronic pain, diagnosing a chronic pain-related abnormal brain condition, locating at least one area of abnormal brain activity associated with the abnormal brain condition and mitigating the abnormal brain activity by applying a neuromodulation signal to tissues corresponding to the at least one area of abnormal brain activity. Alternatively, the neuromodulation signal comprises waveforms designed to minimize tissue impedance while effecting noninvasive neuromodulation. Treatment effect is realized when abnormal brain function has been improved or corrected.

A physical assessment may first be performed of a human subject presenting with a complaint of symptoms characteristic of a chronic pain condition such as fibromyalgia. The physical assessment may include, among other things, a determination of chronic widespread pain, sleep difficulty, fatigue, morning stiffness of the muscles and joints, cognitive difficulty, and other symptoms associated with the condition. The physical assessment may also include tests performed to exclude various non-fibromyalgia conditions as the cause of the symptoms. Such further testing may include palpation of 18 tender points in the manner prescribed by the American College of Rheumatology, with such palpation being performed to determine whether the subject has an abnormal sensitivity to pain.

In the absence of an alternate, non-fibromyalgia diagnosis, an electroencephalogram (EEG) test may be performed in addition to the physical assessment, whereby the EEG test is performed utilizing methods and apparatus well known in the art. Specifically, the subject may be made comfortable by, for example, being seated, or reclined. Preparation of the scalp in accordance with commonly followed procedures for performing a clinical EEG may be done by a person of sufficient competence. EEG electrodes may then be adapted to be worn on the scalp, preferably in scalp locations identified as the “International 10-20” standard sites, using common methods of affixing the electrodes such that they rest on or otherwise contact tissues.

While any number of electrodes may be used, a preferred number is either 19 or 24, in accordance with the number of electrode sites used to construct various independent databases utilized to represent the EEG of a healthy normal population, and to facilitate quantitative assessment (qEEG) of the subject's EEG. Methods involving qEEG include a number of mathematical analyses utilized to make statistical comparisons between the subject's qEEG and a database of qEEGs of either healthy normal individuals' brain functions or the brain functions of individuals suffering from chronic pain related brain function conditions.

Records of the subject's EEG from each electrode site may then be acquired under the conditions of both their eyes being closed and their eyes being open, with each condition producing a separate data record. In other words, an “eyes open” EEG record may be obtained, which includes EEG data obtained from each electrode site while the subject's eyes are open and an “eyes closed” EEG record may be obtained, which includes EEG data obtained from each electrode site while the subject's eyes are closed. Preferably, a minimum of five minutes of EEG data may be obtained from each electrode site for each “eyes open” EEG record and a minimum of five minutes of EEG data may be obtained from each electrode site for each “eyes closed” EEG record to assure that enough EEG data is recorded to produce statistically significant samples from each electrode site, both with the subject's eyes open and with the subject's eyes closed. This is further described below.

Preferably, an additional test may be performed in which at least one additional EEG record is made that includes EEG data obtained at each electrode site while the subject's eyes are closed. In this test, henceforth referred to as a “tender point palpation (TPP) test”, a number of tender points on the subject's body, preferably ranging between one and 18, are identified and serially palpated with an algometer. Preferably, four tender points may be chosen, and, preferably, those four points include tender points adjacent the right and left lateral epicondyle of the arms approximately two centimeters distal of the elbows, and tender points adjacent the right and left costochondral junctions of the second rib.

The TPP test may be executed by acquiring an EEG record (“TPP” EEG record) including EEG data obtained from the electrode sites for a first tender point by first commencing the acquisition of EEG data and then, a short period of time later, commencing palpation of the first tender point. Preferably, the period of time between the commencement of data acquisition and the commencement of palpation of the first tender point may be between one and 300 seconds. Palpation of the first tender point may be accomplished by pressing on the tender point—preferably pressing or palpating through the use of an algometer, and preferably at a rate of approximately one kilogram per centimeter squared per second, until the subject reports a painful sensation or until reaching a pressure of 4 kilograms per centimeter squared—whichever occurs first. Preferably, palpation pressure may be removed as soon as the subject reports a painful sensation. A record is made of the amount of the pressure being applied at the moment the subject reports a painful sensation.

Further according to the TPP test method, the recording of the “eyes closed” EEG may continue for a period of time after release of palpation pressure, preferably between 1 and 300 seconds, and most preferably, for at least 60 seconds. Following this period, a second and subsequent tender point may be serially palpated with an algometer in the same manner as described for the first, with “TPP” EEG records being recorded for each by recording the “eyes closed” EEG for each site in the manner described with regard to obtaining the “TPP” EEG record for the first site. This process may be repeated for each chosen tender point. Accordingly, the resulting EEG data record includes the “TPP” EEG records acquired for each chosen tender point.

The “TPP” EEG records may be acquired for a period of time that is sufficient to extract from each “TPP” EEG record a minimum of 60 seconds of “clean” EEG data, that is, data free of extraneous electrical noise such as that from electromyographic movement. Preferably, all EEG records (“eyes open” EEG records, “eyes closed” EEG records, and “TPP” EEG records) may be individually edited to provide from each EEG record a minimum of 60 seconds of clean EEG. Preferably, the clean data is obtained to present a high degree of statistical consistency. Such measures as “Split-Half” reliability, which is the ratio of variance between the even and odd seconds of the time series of selected clean EEG; and “Test Re-test” reliability, which is the ratio of variance between the first half and the second half of the selected clean EEG segments may be used. Preferably, clean EEG data is obtained such that measures of these ratios are a minimum of 0.95 and 0.90 respectively, which is consistent with levels of reliability commonly published in EEG literature.

With regard to the TPP test method, clean data includes only that EEG data acquired after palpation of a tender point, and does not include any EEG data acquired during the palpation of a tender point. In addition, to assess the stability of a “TPP” EEG record, EEG data acquired before palpation of a tender point may be removed, edited and statistically compared to like data in the “eyes closed” EEG record obtained from the “eyes closed” EEG test. Stability of the “eyes closed” and “TPP” EEG records is indicated by a finding that there is no statistically significant difference between the “eyes closed” EEG record and the pre-palpation portion of the “TPP” EEG record. A contrary finding indicates instability and a need to repeat the EEG tests.

Further to the method, and in the preferred embodiment, clean “eyes open”, “eyes closed”, and “PPT” EEG records may be then mathematically analyzed for various time domain and frequency domain parameters of their respective electrical signals. These analyses may include, but are not limited to voltage analysis, current analysis, voltage and current analysis, frequency spectrum analysis using Fast Fourier transform analysis, frequency spectrum analysis using a wavelet analysis method, frequency spectrum analysis using absolute power analysis method, frequency spectrum analysis using relative power analysis method, frequency spectrum analysis using phase analysis method, frequency spectrum analysis using coherence analysis method, frequency spectrum analysis using amplitude symmetry analysis method, and localization of electrical activity in the brain using inverse EEG computation analysis.

Findings from the aforementioned analyses may then be statistically compared to the same parameters determined from “eyes open”, “eyes closed”, and “PPT” EEG records taken from an age and gender matched database of healthy normal individuals. Such statistical analyses may include, but are not limited to deviations from a standard normal distribution. Findings of statistically significant abnormal deviation, or lack thereof, may then be presented in a graphical or numerical format for analysis by a competent health care professional or person of similar expertise.

EEG abnormalities consistent with those observed in a sample population of fibromyalgia patients may include, but are not limited to one or more of the following: (1) an overall reduction in EEG power across all spectra in either of the “eyes open” or “eyes closed” conditions; (2) statistically significant low EEG power levels in frontal or temporal regions of any of the delta (1-3.5 hertz), theta (4-7.5 hertz) or alpha (8-12 hertz) frequency segments of EEG for the “eyes closed” condition; (3) statistically significant low coherence among the frontal EEG sites for the delta or theta EEG segments in either of the “eyes closed” or “eyes open” conditions; (4) statistically significant high relative beta (12.5-25 hertz) absolute power in the parietal region of the brain for either of the “eyes closed” or “eyes open” conditions. The magnitude of statistical variation considered statistically “significant” may vary depending on the application. For example, in research, a difference between a sample and a population measure generally has to have a p-value of 0.01 or less for the difference to be considered statistically “significant”. However, in clinical application statistically significant differences may be declared with p-values at the 0.1 level or less.

Further EEG abnormalities consistent with those observed in a sample population of fibromyalgia patients, and drawn particularly to the TPP test method, may include but are not limited to a finding of (1) a statistically significant increase in EEG absolute power, particularly in the alpha and beta segments, in the parietal, occipital, and temporal areas of the brain as compared to the “eyes closed” EEG record (“eyes closed” EEG findings without tender point palpation) for the same subject; or (2) a statistically significant increase in coherence in the alpha or beta segment of EEG. The following are the results of tests of the predictive value of TPP sensitivity analysis, obtained when TPP testing was utilized on 19 fibromyalgia patients and compared to TPP testing done on nine healthy normal controls:

| | | | Positive |
|---|---|---|---|
| Test Criterion for Making a Diagnosis of | Sensi- | Speci- | Predictive |
| Fibromyalgia | tivity | ficity | Value |
| An increase* in alpha EEG of at least 20% | 63% | 89% | 92% |
| in at least one occipital or parietal site | | | |
| An increase in alpha EEG of at least 20% | 84% | 78% | 89% |
| in at least one temporal site | | | |
| The total regions of increase in alpha EEG | 74% | 100% | 100% |
| of at least 20% are greater than two | | | |
| Alpha EEG coherence increases by at least | 84% | 88% | 94% |
| 20% in at least 30 out of 171 possible | | | |
| site combinations | | | |
| At least two (2) positive findings occur | 90% | 100% | 100% |
| in any of the four previous tests | | | |

*Based on comparison of TPP EEG data against eyes closed EEG data

A diagnosis of fibromyalgia may be made when physical assessment findings that support a diagnosis of fibromyalgia are augmented by making a quantitative assessment including but not necessarily limited to a statistical comparison between the subject's qEEG and a database of quantitative assessments of either healthy normal individuals or individuals suffering from a chronic pain related abnormal brain function condition such as fibromyalgia. In the preferred embodiment, statistical findings that support a diagnosis of fibromyalgia may include, but are not necessarily limited to, (1) an abnormal finding resulting from the TPP test, preferably a finding of a statistically significant increase in EEG absolute power, and particularly in the alpha and beta segments, in the parietal, occipital, and temporal areas of the brain as compared to the “eyes closed” findings without tender point palpation for the same subject; and preferably (2) an abnormal finding resulting from the “eyes closed” EEG test, preferably statistically significant low EEG power levels in frontal or temporal regions of any of the delta, theta or alpha frequency segments of EEG for the “eyes closed” condition, and most preferably with an additional finding of statistically significant low coherence among the frontal EEG sites for the delta or theta EEG segments. Alternately, fibromyalgia may be diagnosed by statistically comparing a subject's one or more qEEG parameters to like qEEG parameters obtained from at least one healthy normal individual; then comparing the one or more deviations to deviations detected in a sample population of known fibromyalgia patients.

Clean EEG records from a subject may be mathematically analyzed for various time domain and frequency domain parameters of their electrical signals, consistent with analysis techniques already described, and then findings from these mathematical analyses may be statistically compared to like parameters taken from an age and gender matched database of healthy normal individuals or individuals known to have fibromyalgia. The statistical comparisons may include, but are not limited to deviations from a standard normal distribution of like EEG measures associated with members of a database of healthy normal individuals or individuals known to have fibromyalgia. The results of those comparisons may then be presented in a graphical or numerical format for analysis by a competent health care professional or person of similar expertise for the existence of statistically significant abnormal deviations, or the lack thereof. A finding in support of a fibromyalgia diagnosis would be supported if there is an absence of any significant deviation between measures from a subject's clean EEG and those from a database comprising individuals known to have fibromyalgia.

Analyses of clean EEG from a subject may be statistically correlated to measures of symptom severity. As previously described, analysis findings may be mathematically analyzed for various time domain and frequency domain parameters of electrical signals. A number of measures of the magnitude of deviation from standard normal distributions of either healthy normal EEG or known fibromyalgia patient EEG can be determined. The magnitudes may be presumed to be related to the severity of the condition and may be statistically correlated to such symptom measures that may include, but are not limited to, tender point pain pressure thresholds as determined by an algometer, and various other indices of pain derived from the algometry measures (e.g. the sum of all 18 tender point pain tolerance measures, the average of all 18 tender point pain tolerance measures, etc.). Such analysis has utility in both predicting symptom severity in individuals with fibromyalgia, and in determining the effect of therapeutic intervention to correct or manage symptoms of fibromyalgia.

EEG analyses may also be used for determining the location of abnormal brain activity and further for determining points for application of neuromodulation.

Treatment may include the application of a noninvasive neuromodulation signal in a manner designed to correct abnormal brain function identified in accordance with the aforementioned EEG analyses. Suitable noninvasive neuromodulation techniques are disclosed in applicant's U.S. patent application Ser. No. 11/490,255 and applicant's International Patent Application Ser. No. PCT/US2008/72395, which are incorporated herein by reference. The noninvasive neuromodulation signal may comprise those waveforms designed to minimize tissue impedance, such as an “amplitude modulated pulse width modulated” (AMPWM) signal. An AMPWM signal utilizes a high frequency carrier signal that is amplitude modulated by a low frequency neuromodulation signal. The carrier signal is of sufficiently high frequency so as to be less attenuated by the impedance of tissues due to their capacitive reactance. The frequencies used in the neuromodulation signal are lower than the frequency of the carrier signal, and are chosen to provide therapeutic benefit. By using the neuromodulation signal to amplitude modulate the carrier signal, and subsequently applying the combined signal to tissues, the neuromodulation signal is less attenuated by the impedance of the tissue permitting greater penetration of electrical current and field. The carrier signal is further pulse width modulated in an AMPWM signal to control the time averaged current, and hence the power of the signal delivered to the tissues. An apparatus for generating and delivering an AMPWM signal includes any number of electric signal generating devices capable of generating and altering the parameter aspects of an AMPWM signal. Various forms of an AMPWM signal and apparatus for generating an AMPWM signal are disclosed in the applicant's U.S. application Ser. No. 11/490,255 and applicant's International Patent Application Ser. No. PCT/US08/72395. Reports on the results of a double-blind, placebo-controlled study of the efficacy of this treatment are summarized below:
Thirty-nine (39) active treatment (AT) fibromyalgia patients and 38 comparable placebo control patients completed non-invasive neuromodulation treatment, applied twice a week for 11 weeks. The placebo condition (PL) was created by not delivering the non-invasive neuromodulation signal. Both number of tender point (defined by the American College of Rheumatology) and total pain score were evaluated at baseline and end of treatment. Subjects also completed health impact questionnaires (Fibromyalgia Impact Questionnaire [FIQ], Symptom CheckList-90 [SCL-90], Beck Depression Inventory [BDI], and sleep quality) at baseline and end of treatment period, and FIQs in long-term follow up. Primary outcome measures were changes in the number of tender points (TePs) and level of TeP pain, secondary measures were changes in the questionnaire responses.
Analysis of results showed AT patients improved in number of positive TePs, mean 17.4 pre-treatment to 9.9 post-treatment (P<0.001). The between group change was significantly improved (PL −0.2 versus AT −7.4, P<0.001). Sixty-two percent (62%) of the AT group no longer met the tender point criteria for FM classification following treatment. Similarly, the tender point score (TPS) for the AT group improved from 36.7 to 56.4 (P<0.001) whereas the control group got slightly worse, 38.9 to 35.8. The between group change was also significantly improved (PL −3.2 versus AT +19.6, P<0.001). The total FIQ score in the AT group improved from 65.1 to 46.0 (P<0.001). In other measures, the AT group reported 61.8% improvement in sleep quality (P<0.001). Long term follow-up FIQ analysis was done at an average of 16.8 months since discontinuation of treatment (range 12-28 months). There was a continuing long term improvement over baseline values (P<0.001). There were no significant side effects.

Mitigation of abnormal activity is accomplished by generating and applying to the subject an electrical stimulation signal having at least one parameter configured to modulate at least one abnormal aspect of the subject's EEG, which corresponds to at least one statistically significant difference found in the statistical comparison of qEEGs. Such abnormal aspects of the subject's EEG may include, but are not limited to, abnormally high or low amplitudes, abnormal amplitudes in specific frequencies or frequency segments, abnormal spectral power, abnormal relative power, amplitude asymmetry, and abnormally high or low coherence. The application of said electrical stimulation signal, preferably an AMPWM noninvasive neuromodulation signal, may comprise a first step of choosing neuromodulation signal parameters intended to correct abnormal brain function identified in accordance with the aforementioned EEG analyses. These parameters may include, but are not limited to, a choice of the carrier signal frequency, neuromodulation signal frequency, amplitude, waveform, duty cycle, application times, and phase. The step of choosing neuromodulation signal parameters may include identifying a particular signal parameter, such as a frequency from a patient's EEG, that is statistically different than normal, e.g., an EEG frequency that is lower than normal at a particular location. The chosen neuromodulation signal parameters may thus, for example, include a frequency generally equal to that of the abnormally low measured EEG frequency. The step of choosing neuromodulation signal parameters may alternatively or additionally include identifying an area of the brain where the spectral amplitude of an EEG frequency measure is found to be statistically different than normal. This step may further include identifying the direction and magnitude of said spectral amplitude deviation from normal for said statistically different than normal EEG frequency measure. The identifying step may yet further include choosing signal parameters that include frequencies ranging between the frequency of the statistically different than normal measure (F1) and a frequency that is (a) within an approximate range of from 20 Hertz greater than F1 to F1 for the case in which the direction of deviation for F1 is less than normal; or (b) within an approximate range of from 20 Hertz less than F1 to F1 for the case in which the direction of deviation for F1 is greater than normal. The identifying step may further include choosing signal amplitudes and application times that are proportional to the magnitude of deviation from normal for said statistically different than normal EEG frequency measure. The identifying step may further include choosing signal duty cycle so as to provide a signal that cannot be felt by a person when applied. The identifying step may further include choosing a signal waveform that encompasses at least one of the frequencies in the range of F1 plus or minus 20 Hertz. The identifying step may also include applying at least two neuromodulation signals to different areas of the brain, and applying a phase shift between the at least two signals where the phase shift may range between zero and 180 degrees.

The application of a noninvasive neuromodulation signal may further comprise the step of choosing neuromodulation signal application location to provide for application of neuromodulation to tissues corresponding to one or more of the spatial location(s) of abnormal brain function identified by the aforementioned analyses. Signal application may further include the use of electrodes to create a signal path between an electrical stimulation signal source such as an apparatus for generating an AMPWM signal and a stimulating electrode positioned proximate to brain tissues in at least one area of abnormal brain activity, either invasively or non-invasively.

Delivery of the neuromodulation signal may be accomplished by utilizing an electrode set comprising invasive stimulating electrodes positioned on or in near proximity to brain tissues exhibiting abnormal function, i.e., within approximately 20 mm. The electrode set may further comprise an invasive ground electrode positioned such that a vector path between stimulating electrodes and a ground electrode passes through tissues to be stimulated.

Delivery of the neuromodulation signal may be accomplished by utilizing an electrode set comprising one or more non-invasive stimulating electrodes adapted to be worn by a subject such that the stimulating electrodes rest on the scalp in proximity to brain tissues exhibiting abnormal function. The electrode set may further comprise a non-invasive ground electrode adapted to be worn by a subject such that the non-invasive ground electrode rests on the scalp in proximity to brain tissues exhibiting abnormal function; positioned such that a vector path between non-invasive stimulating electrodes and a non-invasive ground electrode passes through or in near proximity to tissues to be stimulated.

Delivery of the neuromodulation signal may be accomplished by utilizing an electrode set comprising one or more non-invasive stimulating electrodes adapted to be worn by a subject such that the stimulating electrodes rest on the skin posterior to the cervical vertebrae and in proximity to the vagus nerve. The electrode set may further comprise a non-invasive ground electrode adapted to be worn by a subject such that the non-invasive ground electrode rests on the scalp in proximity to brain tissues exhibiting abnormal function; positioned such that a vector path between non-invasive stimulating electrodes and a non-invasive ground electrode passes through or in near proximity to tissues to be stimulated.

The period of time over which therapeutic intervention takes place may comprise repeated application of a neuromodulation signal for finite duration, with rest time taking place between applications, and total number of applications comprising a finite number. The finite duration may be between one second and 60 minutes; the rest time may be between one minute and seven days; and the total number of applications may be between one application and 300 applications. The number of applications may be proportional to either the extent of abnormal function and/or the time that the abnormal function has been present

The method of generating described neuromodulation signals may include the use of an apparatus such as that disclosed in the applicant's U.S. patent application Ser. No. 11/490,255, which is assigned to the assignee of the present application and incorporated herein by reference.

The method may include the steps of repeating quantitative assessments such as EEG testing, TPP testing and statistical analysis on a subject, as described herein, following a period of therapeutic intervention on said subject. The method may further comprise statistical comparison of parameters of the repeated statistical analysis to like parameters of the statistical analysis of the subject done before the previous therapeutic intervention was started. Such comparison might include, but is not limited to, paired t-testing statistics, correlation analysis of changes in symptom severity, and subsequent comparison to a database of age and gender matched healthy normal individuals or individuals suffering from a brain related chronic pain condition such as fibromyalgia. These comparisons may be used to assess the effectiveness of the therapeutic intervention, in particular noninvasive neuromodulation, or to determine if an alternate intervention is indicated in the absence of treatment effect from a current therapeutic intervention. The comparisons could also be used to determine if further therapeutic intervention is indicated in the absence of any abnormal findings. The comparisons may further be used to modify neuromodulation signal parameters in accordance with the findings of the repeated quantitative assessment step.

With specific reference to the TPP test, repeat testing may include the application of tender point pressure using, e.g., an algometer, only to the levels required to cause a painful response recorded in the same testing performed before therapeutic intervention.

Further according to the method, EEG data may be acquired at a first location (e.g. a clinical location) and the acquired EEG data transferred via electronic means to another location (e.g. a central analysis location) for the herein described analysis and statistical comparisons to be accomplished. The electronic means of data transfer may include, but isn't limited to means of data transfer across a local area network and/or the Internet. Consequently, analysis and statistical findings may then be transferred from a central analysis location to a clinical location, where they may be used in various ways by a physician or similarly qualified health care professional for the determination of parameters of a neuromodulation signal used for therapeutic intervention and treatment of fibromyalgia.

Further according to the method, EEG data may be acquired at a first location (e.g. a clinical location) and the acquired EEG data transferred via electronic means to another location (e.g. a central analysis location) for a purpose such as increasing the size of various databases of individuals known to be suffering from fibromyalgia, individuals known to be suffering from a chronic pain condition that is not fibromyalgia, and healthy normal individuals.

Further according to the method, neuromodulation signal parameters may be determined at a central analysis location and subsequently transferred as data via electronic means to an apparatus at another location (e.g. a clinical location) provided for delivery of a neuromodulation signal used for therapeutic intervention and treatment of fibromyalgia. The electronic means of data transfer may include, but isn't limited to, means of data transfer across a local area network and/or the Internet.

The steps of application of the neuromodulation signal and repeat measurements and analyses of a subject's EEG may be continued until abnormal brain function, as determined by, for example, EEG analysis, is modulated and/or mitigation or resolution of symptoms of the chronic pain condition (such as fibromyalgia) are achieved.

Alternatively, non-EEG methods of assessing brain function may be utilized to quantify and locate abnormal brain function. Such methods include, but are not limited to positron-emission tomography (PET) scans, magnetic resonance imaging (MRI) testing and single photon emission computed tomography (SPECT) scans.

Alternatively, EEG data may be collected during a therapeutic intervention that includes application of an electrical stimulation signal such as a neuromodulation signal, and that EEG data may be analyzed by real-time computational algorithms such as Fast Fourier Transforms (FFT) to determine various statistics associated with EEG, including but not limited to spectral amplitudes of frequencies comprising said EEG. The statistics may be used to modify parameters of a neuromodulation signal for the purposes of optimizing therapeutic benefit. In a preferred embodiment, EEG data collected during a therapeutic neuromodulation signal application is analyzed for spectral components using an FFT algorithm. A comparison between the frequency of a stimulation signal and the highest spectral amplitude of measured EEG signal is made. If said comparison finds these frequencies to be the same, then a corresponding modification to the neuromodulation signal's frequency would be made.

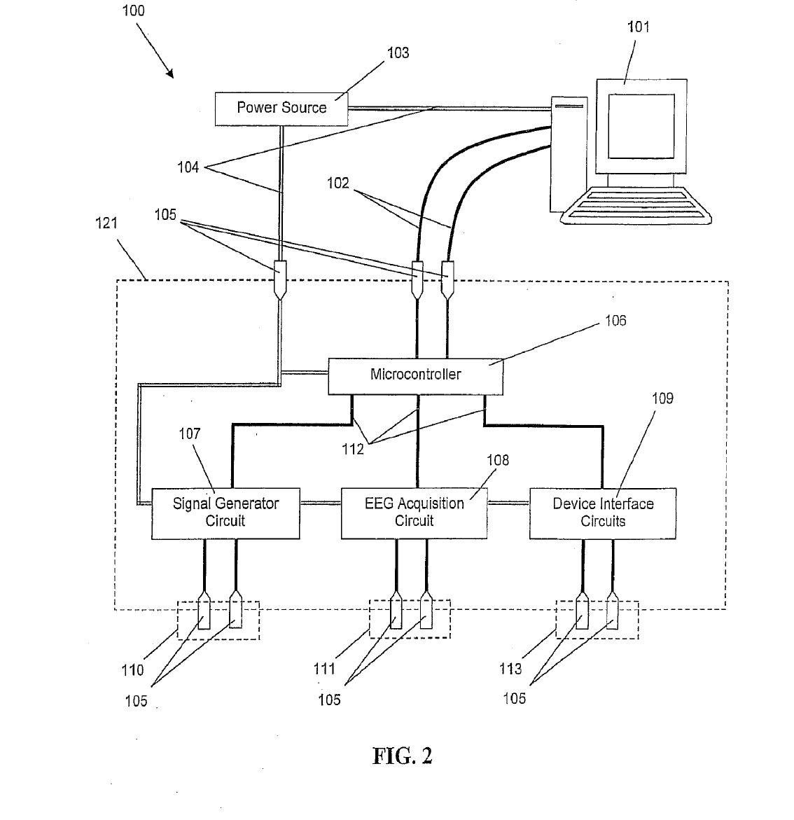

As shown in FIG. 2, the abnormal brain function diagnostic and treatment apparatus 100 may include a computer 101 interfaced to a signal generation and interface module 121 utilizing any number of methods known in the art such as the use of a computer interface cable 102. Any power source 103 known in the art to sufficiently provide power to computers and electronic devices may be utilized and externally interfaced with power wires 104. The signal generation and interface module 121 may include a microcontroller 106 electronically coupled to a signal generator circuit 107, to an EEG acquisition circuit 108 and to any number of device interface circuits 109. All external interfaces may utilize connectors 105 commonly known in the art. All electrical and electronic coupling methods may utilize conductors 112 known in the art.

In practice, the computer 101 may be configured to communicate via interface 102 to the microcontroller 106 for various purposes including the transfer of AMPWM signal parameters and the receipt of EEG data. The computer 101 may include a user interface that allows an operator to monitor and/or influence operation of the diagnostic and treatment apparatus 100.

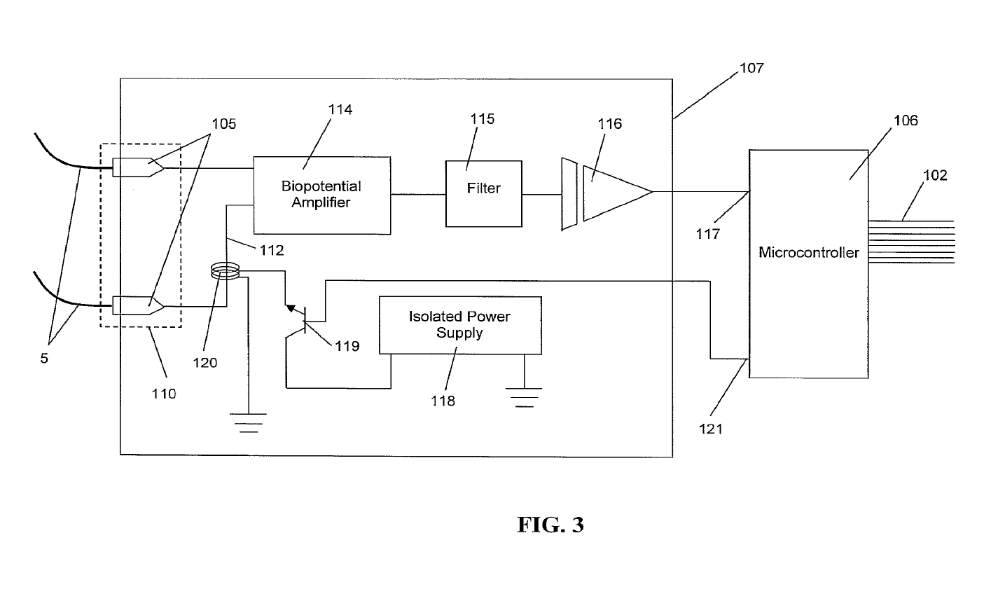

A neuromodulation signal such as an AMPWM signal may be generated in the signal generator circuit 107 and delivered to a stimulation signal interface 110 that includes connectors 105. As shown in FIG. 3, the signal generator circuit 107 may comprise a biopotential amplifier 114 that measures EEG signals and may be operatively coupled to any number of filter 115 circuits configured to reduce extraneous electrical noise in an EEG signal. The biopotential amplifier 114 may be further operatively coupled to an isolation amplifier 116 for human subject protection, and to a microcontroller 106 through an analog-to-digital interface 117. In operation, EEG may be acquired through a stimulation signal interface 110 comprising electrical conductors 5 interfaced at connectors 105. The acquired EEG may be conducted to a biopotential amplifier 114, filter circuits 115, isolation amplifier 116 and to a microcontroller 106 for use such as, but not limited to, in software executed by an interfaced computer 101 for generating and delivering an AMPWM signal. The signal generator circuit 107 may further comprise an isolated power supply 118 configured to provide circuit power and provide human subject protection, a switching transistor 119 that has base connection to a digital-to-analog interface 121 on a microcontroller 106, and an inductor 120 configured and positioned to induce an electrical stimulation signal such as an AMPWM signal into a conductor 112 leading to a connector 105 in a stimulation signal interface 110. In operation, the microcontroller 106 may generate a stimulation signal and conduct that signal via a digital-to-analog interface 121 to the base of the switching transistor 119. Electrical power from an isolated power supply 118 may then switched on and off through the switching transistor 119 creating an amplified stimulation signal in accordance with the stimulation signal waveform generated by the microcontroller 106. The amplified stimulation signal may be further conducted to an inductor 120, and further induced into a conductor 112 creating a therapy stimulation signal in the conductor 112. The therapy stimulation signal may then be delivered to a human subject via the conductor 112 to a stimulation signal interface 110 comprising electrical conductors 5 interfaced at connectors 105. In other words, the neuromodulation signal may be applied using an apparatus comprising a microcontroller 106 configured to generate signal waveforms and coupled to a signal generator circuit 107 configured to transform the signal waveforms into desired AMPWM neuromodulation signals. The signal generator circuit 107 may comprise circuit elements such as a biopotential amplifier 114 configured to measure EEG signals, a filter circuit 115 configured to reduce electrical noise in EEG signals, an isolation amplifier 116 configured to protect human subjects, an analog-to-digital interface 117 configured to convert analog EEG signals to digital signals, an isolated power supply 118 configured to provide circuit power and human subject protection, a switching transistor 119 configured to generate an amplified stimulation signal by switching on and off electrical power from the isolated power supply in response to stimulation signals received at a base of the switching transistor from the microcontroller, and an inductor 120 configured to induce an electrical stimulation signal into a conductor 112. Additional forms of an AMPWM signal and apparatus for generating an AMPWM signal are disclosed in the applicant's U.S. patent application Ser. No. 11/490,255, which is incorporated herein by reference in its entirety.

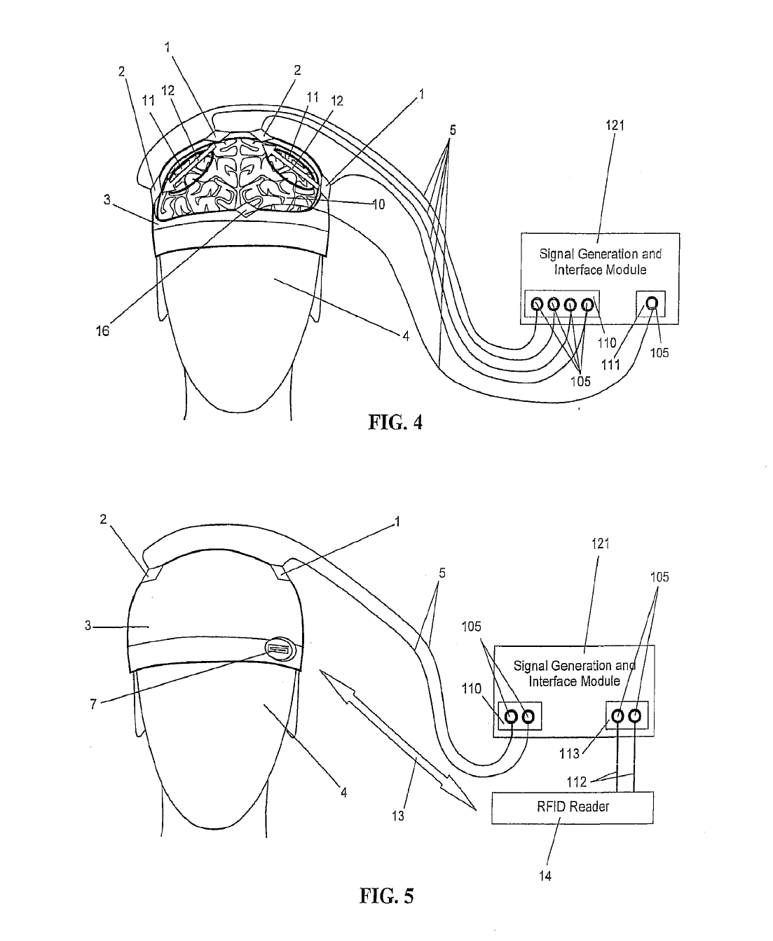

As shown in FIG. 4, the apparatus 100 may include a cap 3 configured to be worn on a subject's head in a predetermined orientation. At least two electrodes 1, 2, which may be non-invasive type electrodes, may be carried by the cap. One of the electrodes 1 may be configured to act as a stimulating electrode 1 for delivering a neuromodulation signal to the subject's head 4, and the other of the electrodes 2 may be configured to act as a ground electrode and to receive neuromodulation signals transmitted by the signal delivery electrode 1. The cap 3 may be of any suitable configuration to include a skull-cap configuration as shown in the drawings, or may simply comprise flexible bands. In any case, the cap 3 is adapted to carry the electrodes 1, 2 and to be worn on the head 4 during mitigation of abnormal brain activity, and, more particularly, to facilitate non-invasive neuromodulation signal delivery to a subject's brain.

EEG from a subject may be collected through the EEG acquisition circuit 108, which may include any form of EEG amplifier instrument known in the art, through an EEG interface 111 that may include connectors 105. At least one additional electrode 16 may be carried by the cap 3 and positioned to sense and transmit EEG signals to the EEG acquisition circuit 108. Alternatively, a stimulating electrode 1 may also serve as an EEG sensor. In other words, one or more stimulating electrodes 1 may be coupled to the EEG acquisition circuit 108 and configured to sense and transmit EEG signals to the EEG acquisition circuit 108. The cap 3 may also carry electrical conductors 5 that provide signal paths between an electrical stimulation signal source such as the signal generator circuit 107 of the diagnostic and treatment apparatus 100 and stimulating electrodes 1 and ground electrodes 2; and the electrical conductors 5 may also provide signal paths between an EEG acquisition circuit 108 and additional electrodes 16, whereby the conductors may electrically couple to connectors 105 at a stimulation signal interface 110 and an EEG interface 111 of a diagnostic and treatment apparatus 100. The stimulating electrodes 1 and ground electrodes 2 may be permanently or removably affixed into the cap 3 in cap locations where, when the cap 3 is placed on a subject's head 4 in a predetermined orientation, the stimulating electrodes 1 and ground electrodes 2 are positioned proximate to respective areas 11 of brain tissues to be stimulated, e.g., areas of brain tissue associated with abnormal brain activity.

The electrodes 1, 2 may be permanently or removably supported in cap locations on the cap 3 so that, when the cap is worn on a subject's head in a predetermined orientation, a vector path 12 extending between the stimulating electrode 1 and the ground electrode 2 passes through the desired area 11 of brain tissues to be stimulated. Further, the cap 3 may be sized in various ways to fit or to be adjustable to a variety of sizes and shapes of human heads 4 and to carry any number of stimulating electrodes 1 and ground electrodes 2 in cap locations that will cause neuromodulation signals to pass along vector paths 12 through predetermined locations of abnormal brain activity 11 in a subject's brain 10. The electrodes 1, 2 may subsequently be removed and placed in new cap locations that will cause neuromodulation signals to pass along vector paths 12 through predetermined locations of abnormal brain activity 11 in a second subject's brain 10.

The cap 3 may be configured to carry any number of electrical circuits known in the art for storing information. As shown in FIG. 5, such circuit may include a radio frequency identification (RFID) chip 7 incorporated into a cap 3 and utilized to store information including, but not limited to, the identification of the subject the cap is intended to be used on, dates and times of use, parameters of an electrical stimulation signal to be used in association with the cap and delivery of non-invasive neuromodulation, a total number of times the cap has been used and monitoring data associated with quality of use.

The diagnostic and treatment apparatus 100 may include any number of external devices that may be utilized in the process of providing assessment, diagnostics, or therapy and that may be coupled to the device interface circuit 109 and interfaced through a device interface 113 that may include connectors 105. For example, the apparatus 100 may include an RFID reader 14 for establishing electrical connectivity between the microcontroller 106 and the RFID chip 7 through a device interface 113. The use of an RFID reader 14 and RFID chip 7 creates a radio frequency pathway 13 that allows information incorporated into the RFID chip 7 may be accessed and utilized by software executed by the microcontroller 106 and/or an interfaced computer 101.

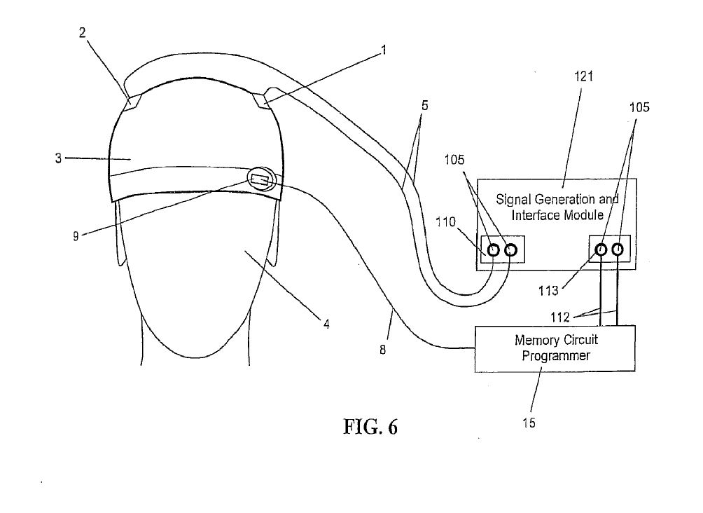

As shown in the embodiment illustrated in FIG. 6, as an alternative to the use of an RFID chip 7, other suitable methods known in the art for accessing information stored in electrical circuits may be used, such as methods that use direct electrical connections via conductors such as wires 8. Such methods may include the use of any number of programmable memory circuits 9 connected via wires 8 to a memory circuit programmer 15, which may be further interfaced to the microcontroller 106 and/or the computer 101 through the device interface 113, such that information incorporated into the programmable memory circuit 9 may be accessed and utilized by the microcontroller 106 and/or the computer 101 via, for example, software executed by the computer 101.

Alternatively, neuromodulation may be used as a method of treatment for fibromyalgia in combination with treatment of other coexisting physical conditions that may or may not be associated with fibromyalgia. In addition, or alternatively, neuromodulation may be used as a method of treatment in combination with other forms of treatment utilized to affect symptoms of fibromyalgia.

1. A method for treating brain-related chronic pain disorders in human subjects, the method including the steps of:
assessing the brain function of a subject suffering from chronic pain;
diagnosing a chronic pain-related abnormal brain condition;
locating at least one area of abnormal brain activity associated with the abnormal brain condition; and
mitigating the abnormal brain activity by applying an electrical stimulation signal to tissues corresponding to the at least one area of abnormal brain activity.

2. The method of claim 1 in which the electrical stimulation signal comprises waveforms configured to minimize tissue impedance.

3. The method of claim 2 in which the electrical stimulation signal comprises an AMPWM signal.

4. The method of claim 1 in which:
at least one of the assessing and locating steps includes obtaining an electroencephalogram (EEG) of the subject's electrical brain activity;
the diagnosing step includes obtaining a quantitative assessment (qEEG) of the subject's EEG and making a statistical comparison between the subject's qEEG and a database of qEEGs of either healthy normal individuals' brain functions or the brain functions of individuals suffering from the chronic pain-related abnormal brain function condition; and
the mitigating step includes generating and applying to the subject an electrical stimulation signal having at least one parameter configured to modulate at least one abnormal aspect of the subject's EEG, which corresponds to at least one statistically significant difference found in the statistical comparison of qEEGs.

5-7. (canceled)

8. The method of claim 1 in which:
the diagnosing step includes diagnosing fibromyalgia by making a statistical comparison between the subject's qEEG and a database of qEEGs of either normal, healthy individuals or individuals suffering from fibromyalgia, and
the mitigating step includes generating and applying to the subject a neuromodulation signal.

9. The method of claim 8 in which at least one neuromodulation signal parameter to be configured for modulation is selected from the group of parameters consisting of carrier signal frequency, neuromodulation signal frequency, amplitude, waveform, duty cycle, application times, and phase.

10. The method of claim 8 in which the neuromodulation signal is applied using an apparatus comprising:
a signal generation and interface module (121) including a microcontroller (106) configured to generate signal waveforms and coupled to a signal generator circuit (107) configured to transform the signal waveforms into desired AMPWM neuromodulation signals;
a cap (3) configured to be worn on a subject's head and carrying first and second electrodes (1, 2) in respective cap locations that, when the cap is placed on a subject's head, position the electrodes adjacent a predetermined area of brain tissues (11) to be stimulated; and
electrical conductors (5) that provide signal paths between the signal generation and interface module (121) and the cap (3).

11. The method of claim 8 in which:
EEG data is collected during neuromodulation signal application;
at least one statistic associated with the collected EEG data is determined by analyzing the collected EEG data using at least one real-time computational algorithm; and
at least one electrical stimulation signal parameter is modified in response to the at least one statistic.

12. The method of claim 8 in which the neuromodulation signal is applied repeatedly, alternating with rest periods.

13-15. (canceled)

16. The method of claim 8 in which:
at least one of the assessing and locating steps includes obtaining an electroencephalogram (EEG) of the subject's electrical brain activity;
the diagnosing step includes obtaining a quantitative assessment (qEEG) of the subject's EEG and making a statistical comparison between the subject's qEEG and a database of qEEGs of either healthy normal individuals' brain functions or the brain functions of individuals suffering from the chronic pain-related abnormal brain function condition; and
the mitigating step includes generating and applying to the subject a neuromodulation signal having at least one parameter configured to modulate at least one abnormal aspect of the subject's EEG, which corresponds to at least one statistically significant difference found in the statistical comparison of qEEGs,
the assessing and quantitative assessment steps are repeated at least once; and
the neuromodulation signal parameters are modified in accordance with the findings of the repeated quantitative assessment step.

17. The method of claim 12 in which repeated applications of neuromodulation signals are continued until the subject's abnormal brain function has been modulated.

18. The method of claim 12 in which repeated applications of neuromodulation signals are continued until the subject's symptoms have been mitigated.

19-33. (canceled)

34. The method of claim 1 in which at least one of the assessing and locating steps includes:
collecting EEG, MRI, PET, or SPECT data during electrical stimulation signal application;
determining at least one statistic associated with the collected EEG, MRI, PET, or SPECT data by analyzing the collected EEG data using at least one real-time computational algorithm; and
modifying at least one electrical stimulation signal parameter in response to the at least one statistic.

35. The method of claim 1 in which:
the diagnosing step includes obtaining a quantitative assessment of the subject's brain function; and
identifying any abnormal brain activity by making a statistical comparison between the subject's quantitative brain function assessment and a database of quantitative assessments of either healthy normal individuals' brain functions or the brain functions of individuals suffering from the chronic pain-related abnormal brain function condition.

36-55. (canceled)

56. The method of claim 1 in which the mitigating step includes mitigating abnormal brain activity by applying a neuromodulation signal to tissues corresponding to the at least one area of abnormal brain activity.

57. (canceled)

58. (canceled)

59. The method of claim 56 in which the mitigating step includes:
applying a neuromodulation signal along a signal path between an electrical stimulation signal source and a stimulating electrode; and
positioning the stimulating electrode proximate brain tissues in the at least one area of abnormal brain activity.

60. The method of claim 56 in which the mitigating step includes:
applying a neuromodulation signal along a signal path between an electrical stimulation signal source and a stimulating electrode; and
positioning the stimulating electrode on the subject's scalp proximate brain tissues in the at least one area of abnormal brain activity.

61. The method of claim 60 in which the mitigating step includes positioning a ground electrode such that a vector path between the stimulating electrode and the ground electrode passes proximate tissues in the at least one area of abnormal brain activity.

62. The method of claim 60 in which the mitigating step includes positioning a ground electrode such that a vector path between the stimulating electrode and the ground electrode passes through tissues in the at least one area of abnormal brain activity.

63-70. (canceled)

71. The method of claim 12 in which the step of applying the electrical stimulation signal repeatedly alternating with rest periods includes:
repeating the assessing and quantitative assessment steps at least once following electrical stimulation signal application; and
reapplying the electrical stimulation signals with signal parameters modified in accordance with the findings of the repeated qualitative assessment step.

72-105. (canceled)
